(19) United States
(12) Patent Application Publication
(10) Pub. No.: US 2023/0047766 A1
(43) Pub. Date: Feb. 16, 2023

(54) MEDICAL DEVICE, THERAPEUTIC METHOD, AND DIAGNOSTIC METHODS FOR THE TREATMENT AND PREVENTION OF VASOSPASM

(71) Applicant: Sylvian Inc., Tokyo (JP)

(72) Inventor: Victor Stone, Boston, MA (US)

(21) Appl. No.: 17/797,785

(22) PCT Filed: Feb. 5, 2021
(86) PCT No.: PCT/IB2021/050962

§ 371 Date: Aug. 5, 2022

Related U.S. Application Data
(60) Provisional application No. 62/971,945, filed on Feb. 8, 2020.

(51) Int. Cl. A61K 31/7016; A61P 25/28; G01N 30/72; A61P 9/10

(52) U.S. Cl. CPC A61K 31/7016; A61P 25/28; G01N 30/72; A61P 9/10

(57) ABSTRACT

A method for treating vasospasm may include measuring cerebrospinal fluid (CSF) to obtain a baseline biomarker value. The method may include administering a first dose of a trehalose solution. The method may include draining the CSF to maintain a current intracranial pressure (ICP). The method may include measuring a trehalose concentration in the CSF. The method may include measuring a biomarker value in the CSF. The method may end based on a determination that the measured biomarker value indicates a predetermined biomarker concentration.

### CROSS-REFERENCE TO RELATED APPLICATION

This application claims priority to and the benefit of U.S. Provisional Application Patent Ser. No. 62/971,945, filed Feb. 8, 2020, the entire disclosure of which is hereby incorporated by reference.

### TECHNICAL FIELD

This disclosure relates to a medical device and method of treatment.

### BACKGROUND

Berry Aneurysm is a disease affecting the arteries (blood vessels) in the brain. Small weaknesses in the arterial wall result in a balloon-like structure characterized with a thin wall localized in specific areas deep in the brain. Arterial blood pressure creates a continuous strain on the thin walls, and the aneurysm continues to grow, with a constant probability of rupture. The rupture of berry aneurysms results in Subarachnoid Hemorrhage (SAH), otherwise known as “wet-stroke.” While berry aneurysms are relatively benign conditions, SAH is a condition that is sudden onset and causes devastating results to the patient.

When an aneurysm ruptures, significant amounts of blood floods out of the rupture site into cisterns (caves) within the brain, called ventricles. This abnormal course of blood results in two problems: (a) brain matter distal to the rupture site cannot receive blood and suffers ischemia (lack of blood) with ensuing neural damage, and (b) whole blood in ventricles causes a cascade of inflammatory reactions. One major outcome of (b) is a spasm of arteries (vasospasm) causing delayed cerebral ischemia (DCI) to a wider area of the brain 4-5 days following initial rupture. It is important to note that berry aneurism rupture causes immediate ischemic damage to the brain, and vasospasm causes delayed ischemic damage to a broader area of the brain.

To treat SAH, the ruptured aneurysm must be repaired, either by open surgery (surgeon exposes the ruptured artery and secures it with a clip) or through endovascular means (a coil or clot forming scaffold material is placed inside the berry aneurysm to secure the rupture by clot formation). Neurosurgery saw significant advancement in treatment modalities to secure the ruptured site. However, the treatment of complications resulting from hemorrhaged blood in ventricles has not seen significant advancement.

Numerous therapies were attempted to treat vasospasm (post securement of aneurysm) and prevent DCI. Conventional therapies include the legacy Triple-H therapy (hypertension, hypervolemia, and hemodilution) and calcium channel blocker, nimodipine. Clinical trial databases show various attempts with novel compounds such as Clozasentan (ET-1 inhibitor), an intravenous small molecule therapy which demonstrated reversal of vasospasms in large arteries.

### SUMMARY

Disclosed herein are implementations of a medical device, therapeutic method, and diagnostic methods for the treatment and prevention of vasospasm. In an aspect, a method for treating vasospasm may include measuring cerebrospinal fluid (CSF) to obtain a baseline biomarker value. The method may include administering a first dose of a trehalose solution. The method may include draining the CSF to maintain a current intracranial pressure (ICP). The method may include measuring a trehalose concentration in the CSF. The method may include measuring a biomarker value in the CSF. The method may end based on a determination that the measured biomarker value indicates a predetermined biomarker concentration.

In one or more aspects, the method may further include determining whether a predetermined trehalose concentration is reached. In one or more aspects, the method may include administering a second dose of the trehalose solution. The second dose of trehalose solution may be administered on a condition that the predetermined trehalose concentration is not reached. The method may include further administration of trehalose solution.

In one or more aspects, the administering and the draining may be performed simultaneously. In one or more aspects, the administering and the draining may be alternately performed. In one or more aspects, the method may be performed using a brain drainage system. In one or more aspects, the brain drainage system may be a single lumen catheter or a dual lumen catheter.

In one or more aspects, the trehalose solution may be approximately a 5 wt % to 40 wt % trehalose solution. In one or more aspects, the measured trehalose concentration in the CSF may be within a therapeutic range. The therapeutic range may be about 7 wt % to about 10 wt %. In one or more aspects, the trehalose solution may be administered at a rate based on the metabolism rate of a subject. In one or more aspects, the measured biomarker value may be an inflammatory marker value or a blood metabolite value. In one or more aspects, the inflammatory marker value may include an interleukin-2 (IL-2) concentration, a tumor necrosis factor (TNF) concentration, any cytokine concentration, or any combination thereof. In one or more aspects, the blood metabolite value may include a bilirubin concentration or a metabolic intermediate of red blood cells.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a flow diagram of an example of a method of use to treat vasospasm in accordance with embodiments of this disclosure.

FIG. 2 is a diagram of an example of a method of use to treat vasospasm in accordance with embodiments of this disclosure.

FIG. 3 is a graph showing the concentrations of trehalose and a biomarker during the treatment of vasospasm in accordance with embodiments of this disclosure.

FIGS. 4A-4G are graphs showing simulations of trehalose concentrations in human CSF to determine clinical dose regimens.

### DETAILED DESCRIPTION

Trehalose may be used as a therapeutic or prophylactic agent for vasoconstriction as described in U.S. Pat. No. 8,283,337, the contents of which are hereby incorporated by reference. As demonstrated in the Examples below, the agent in accordance with the embodiments described herein comprise trehalose as the active ingredient for the treatment and prevention of vasospasm. It may be possible to prevent vasospasm effectively by using the agent as a perfusion agent comprising an effective concentration of trehalose.

Vasospasm induces ischemia such as cerebral ischemia. The agent may prevent ischemia and may be used as an improver for preventing or reducing the progress of ischemia. The improver may be effective for the treatment and/or prevention of ischemia, such as for example, cerebral ischemia.

Vasospasm induces cerebral infarction. The agent may prevent cerebral infarction and may be used as an improver for preventing or reducing the progress of cerebral infarction. The improver may be effective for the treatment and/or prevention of cerebral infarction.

Trehalose is a disaccharide compound (2 sugar molecules bound together), commonly seen as a food preservative. The compound has cytoprotective (protects cells) effects by creating a superficial coat on tissue and stabilizing cell membranes. Shimohata et al. report potential therapeutic effects of Trehalose pertaining to SAH. (See Shimohata, et at, “Trehalose decreases blood clotting in the cerebral space after experimental subarachnoid hemorrhage.” J Vet Med Sci. 2020 May; 82(5): 566-570). When trehalose is placed into cranial (brain) ventricles in high concentrations (˜7%), trehalose replaces whole blood on arterial surfaces, as well as create a coating around blood components including platelets. Animal studies show a significant inhibition of the inflammatory cascade and vasospasm associated with SAH. Trehalose has potential to prevent vasospasm and delayed cerebral ischemia (DCI).

Cranial ventricles are not readily accessible, however, may be accessible via catheterization for treatment of advanced stage SAH. An opening in scalp, cranium, and dura is made locally, and a catheter is coursed through brain matter to access the lateral ventricle or other cisternal or subdural space. The catheter may be a dual lumen catheter with ability to irrigate (administer) fluids and suction (extract) cerebrospinal fluid (CSF) simultaneously.

The content of trehalose may be adjusted based on the application, form of formulation, patient, or any combination thereof. If the agent is a liquid formulation for intracranial administration, the content of trehalose is preferably from 5 wt % to 40 wt %, in the agent, and more preferably, it is administered in an amount adjusted to a CSF trehalose concentration of about 2 wt % to about 12 wt %. More preferably, the agent may be administered such that the CSF trehalose concentration is about 7 wt % to about 10 wt %.

The embodiments disclosed herein may include draining CSF as it is synthesized by the body. For example, the trehalose may be introduced such that it permeates into the lateral ventricle and vasculature of the brain. As the trehalose breaks down, it may be drained via the brain drainage system. The blood breaks down and the hemolysate permeates, causing further breakdown. The blood may then be drained, and the hemolysate on the artery causes an inflammatory response. The trehalose replaces the hemolysate and inflammation is reduced.

The embodiments disclosed herein may include administering a tracer compound into the lateral ventricle such that it permeates the vasculature of the brain. The tracer compound may be a molecule that serves as a proxy for trehalose. For example, the tracer compound may be a fluorescent dye such as IVIS or a radiographic compound that may be used in positron emission tomography (PET). The tracer compound may be drained and measured. The tracer compound may be attached to the trehalose molecule.

The embodiments disclosed herein may include administering trehalose and the tracer compound into the CSF of the lateral ventricle via a dual lumen catheter. In this example, the dual lumen catheter may be used to suction CSF simultaneously such that ICP is maintained.

The embodiments disclosed herein may include administering trehalose into the CSF of the lateral ventricle via a single lumen catheter. In this example, the CSF may be suctioned intermittently via the single lumen catheter to maintain ICP.

The embodiments disclosed herein may assume the following drug metabolism and pharmacokinetics (DMPK) factors. For example, soluble compounds and biomarkers may achieve equilibrium within the CSF in a given period of time simulated from animal models. As human intrathecal systems lack trehalase, trehalose break down into monosaccharaides in CSF may occur at a miniscule rate. The majority of intrathecal trehalose clearance may occur by flow from the CSF to systemic vasculature, followed by metabolism in the systemic vasculature.

A sustained CSF concentration simulated from animal models may produce a therapeutic effect. The therapeutic effect may be tracked by computerized tomography (CT), magnetic resonance (MR), cranial doppler, or any combination thereof. The brain may tolerate CSF fluctuations at a range simulated from animal models without causing significant damage.

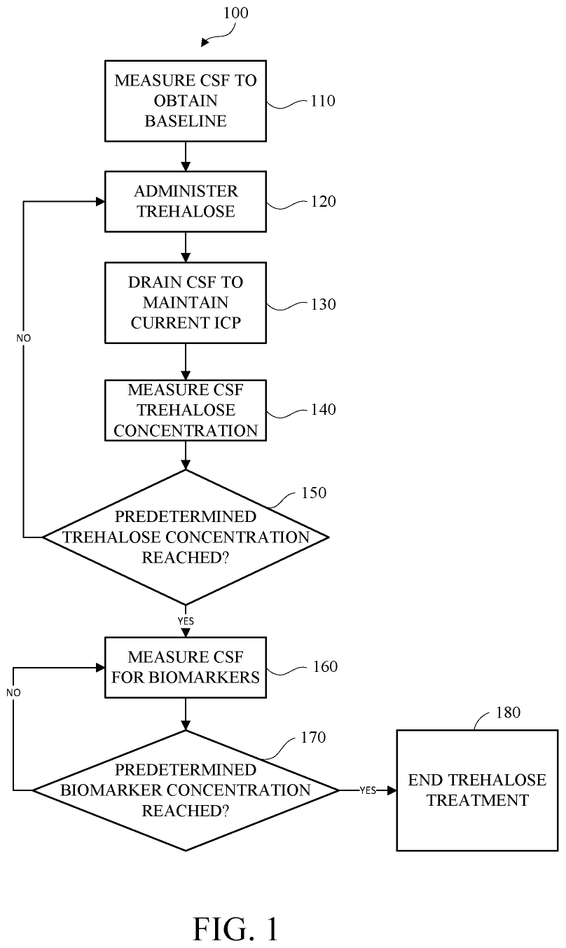

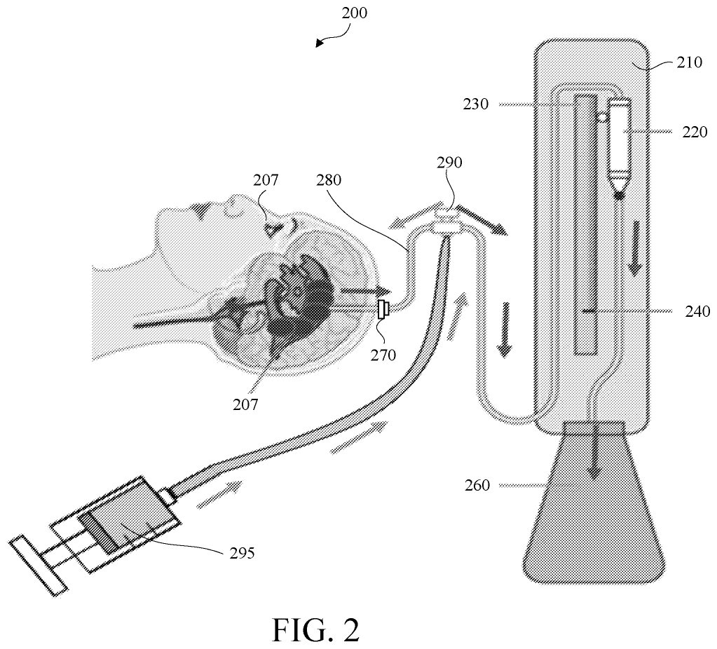

FIG. 1 is a flow diagram of an example of a method 100 to treat vasospasm in accordance with embodiments of this disclosure. The method 100 may be performed following a subarachnoid hemorrhage to prevent DCI. The method 100 includes measuring 110 CSF to obtain baseline biomarker values. The biomarker values may be used to detect inflammation, infection, or both. The biomarker values may include inflammatory marker values, blood metabolite values, or both. Example inflammatory markers may include eukaryotic translation initiation factor 4E (4EBP1), adenosine deaminase (ADA), artemin (ARTN), AXIN1, brain-derived neurotrophic factor (BDNF), beta nerve growth factor (BetaNGF), CASP8, C-C motif chemokine ligand (CCL)11, CCL13/monocyte chemoattractant protein (MCP)4, CCL19, CCL2/MCP1, CCL20, CCL23, CCL25, CCL28, CCL3/macrophage inflammatory protein (MIP)1alpha, CCL4, CCL7/MCP3, CCL8/MCP2, cluster of differentiation (CD)244, CD40, CD5, CD6, CDCP1, colony stimulating factor (CSF)1, CST5, CX3CL1, CXCL1, CXCL10, CXCL11, CXCL5, CXCL6, CXCL9, DNER, EN-RAGE, fibroblast growth factor (FGF)19, FGF21, FGF23, FGF5, FMS-like tyrosine kinase 3 ligand (FLT3L), glial cell line-derived neurotrophic factor (GDNF), hepatocyte growth factor (HGF), interferon (IFN) gamma, interleukin (IL)10, IL10RA, IL10RB, IL12B, IL13, IL15RA, IL17A, IL17C, IL18, IL18R1, IL1alpha, IL2, IL20, IL20RA, IL22RA1, IL24, IL2RB, IL33, IL4, IL5, IL6, IL7, IL8/CXCL8, KITLG/SCF, leukemia inhibitory factor (LIF), LIF receptor (LIFR), lymphotoxin alpha (LTA)/tumor necrosis factor (TNF)B, matrix metallopeptidase (MMP)1, MMP10, neurturin (NRTN), neurotrophin (NTF)3/NT3, oncostatin M (OSM), programmed death-ligand (PDL)1, plasminogen activator urokinase (PLAU)/uPA, SIRT2, signaling lymphocytic activation molecule family member (SLAMF)1, STAMBP, SULT1A1/ST1A1, TGFalpha, TGFB1/, TNF, TNFRSF11B/OPG, TNFRSF9, TNFSF10/TRAIL, TNFSF11/TRANCE, TNFSF12/TWEAK, TNFSF14, thymic stromal lymphopoietin (TSLP), vascular endothelial growth factor (VEGF)A, or any other known molecule related to inflammatory response. Example blood metabolites may include bilirubin and metabolic intermediates of blood components. For example, blood metabolites may include hemoglobin, biliverdin, carbon monoxide (CO), free ferrous iron (Fell), NF-kB, endothelial cell adhesion molecule (ECAM), vascular cell adhesion molecule-1 (VCAM-1), intracellular cell adhesion molecule-1 (ICAM-1), P-selectin, haptoglobin, hemopexin, or phycocyanobilin related molecules. The CSF may be obtained using a brain drainage system such as a single lumen catheter, a dual lumen catheter, or the brain drainage system shown in FIG. 2.

The method 100 includes administering 120 a trehalose solution into the brain, for example the lateral ventricle. The trehalose solution may be administered into the brain using the brain drainage system. The concentration of trehalose administered may be approximately a 5 wt % to 40 wt % trehalose solution. The rate of administration of the trehalose solution may be dependent on the metabolism of the individual subject to metabolize the trehalose.

The method 100 includes draining 130 CSF via the brain drainage system to maintain the current intracranial pressure (ICP). In some embodiments, the CSF may be drained simultaneously as the trehalose solution is administered. In some embodiments the administration of the trehalose solution and the drainage of the CSF may be performed in an alternating fashion.

The method 100 includes measuring 140 the trehalose concentration in the drained CSF. The trehalose concentration may be measured by assays, including mass spectrometry, for example.

The method 100 includes determining 150 whether a predetermined concentration of trehalose has been reached in the CSF. The predetermined concentration of trehalose may be associated with a therapeutic concentration range of trehalose in the CSF. For example, the therapeutic concentration range of trehalose in the CSF may be from about 7 wt % to about 10 wt %. The trehalose concentration in the CSF may be periodically or continuously monitored to determine whether the predetermined concentration is achieved and/or maintained. If the predetermined concentration of trehalose is not reached, the method 100 includes administering 120 trehalose solution into the brain.

If the predetermined concentration of trehalose is reached, the method 100 includes measuring 160 the CSF for biomarkers. The method 100 includes determining 170 whether a predetermined biomarker concentration in the CSF is reached. The predetermined biomarker concentration may indicate that, in the CSF, there is either an acceptable amount or no amount of inflammation, infection, blood metabolites, or any combination thereof. If the predetermined biomarker concentration is not reached, the method 100 continues to maintain the predetermined trehalose concentration in the CSF and measure 160 the CSF for biomarkers. The CSF may be periodically or continuously monitored for biomarkers during treatment. If the predetermined biomarker concentration is reached, the trehalose treatment may end 180.

FIG. 2 is a diagram of an example of method 200 to treat vasospasm in accordance with embodiments of this disclosure. The method 200 may be performed approximately 72 hours after the clipping or coiling of an aneurysm to prevent DCI. As shown in FIG. 2, a subject 205 experiencing a subarachnoid hemorrhage has blood in the lateral ventricle 207.

As shown in FIG. 2, the method 200 includes using a brain drainage system 210. The brain drainage system 210 is configured to maintain the current ICP. In some embodiments, the brain drainage system 210 may be a single lumen catheter or a dual lumen catheter. As shown in FIG. 2, the brain drainage system 210 may comprise a collection chamber 220 and a pressure setting component 230. The pressure setting component 230 includes an indicator 240 that indicates zero on the pressure scale. The indicator 240 may be positioned such that it is horizontally level with the tragus of the ear of the subject 250. The brain drainage system 210 includes a drainage bag 260.

The brain drainage system 210 is attached to an access port 270 via a tube 280. The access port 270 may be a ventricular catheter and may be inserted into the lateral ventricle 207 of the subject 205. The access port 270 enables access to the lateral ventricle and is secured to the scalp of the subject 205. The tube 280 may include a transparent portion. The tube 280 may be secured to the access port 270 such that a portion of the tube is positioned vertically higher than the scalp of the subject 205.

The clamp 290 may be opened to drain CSF from the lateral ventricle 207 and maintain ICP. The CSF may be drained via the tube 280 to the collection chamber 220, and then finally drained into the collection bag 260. In some embodiments, the tube 280 may be opened to the air with a portion of the tube 280 positioned higher than the scalp of the subject 205. In an embodiment where the tube 280 is opened to the air, the vertical distance between the lateral ventricle 207 and the fluid/air border near the open end of the tube 280 approximates the ICP. In some embodiments, the fluid/air border may be represented by the indicator 240.

The drained CSF may be measured to obtain baseline biomarker values. The biomarker values may be used to detect inflammation, infection, or both. The biomarker values may include inflammatory marker values, blood metabolite values, or both. Example inflammatory markers may include 4EBP1, ADA, ARTN, AXIN1, BDNF, BetaNGF, CASP8, CCL11, CCL13/MCP4, CCL19, CCL2/MCP1, CCL20, CCL23, CCL25, CCL28, CCL3/MIP1alpha, CCL4, CCL7/MCP3, CCL8/MCP2, CD244, CD40, CD5, CD6, CDCP1, CSF1, CST5, CX3CL1, CXCL1, CXCL10, CXCL11, CXCL5, CXCL6, CXCL9, DNER, EN-RAGE, FGF19, FGF21, FGF23, FGF5, FLT3L, GDNF, HGF, IFN gamma, IL10, IL10RA, IL10RB, IL12B, IL13, IL15RA, IL17A, IL17C, IL18, IL18R1, IL1alpha, IL2, IL20, IL20RA, IL22RA1, IL24, IL2RB, IL33, IL4, IL5, IL6, IL7, IL8/CXCL8, KITLG/SCF, LIF, LIFR, LTA/TNFB, MMP1, MMP10, NRTN, NTF3/NT3, OSM, PDL1, PLAU/uPA, SIRT2, SLAMF1, STAMBP, SULT1A1/ST1A1, TGFalpha, TGFB1/, TNF, TNFRSF11B/OPG, TNFRSF9, TNFSF10/TRAIL, TNFSF11/TRANCE, TNFSF12/TWEAK, TNFSF14, TSLP, VEGFA, or any other known molecule related to inflammatory response. Example blood metabolites may include hemoglobin, biliverdin, CO, Fell, NF-kB, ECAM, VCAM-1, ICAM-1, P-selectin, haptoglobin, hemopexin, or phycocyanobilin related molecules.

As shown in FIG. 2, a trehalose solution 295 may be administered into the lateral ventricle 207 via the tube 280. The clamp 290 may be applied to seal the tube 280 such that the trehalose solution 295 may be administered into the lateral ventricle 207. The trehalose solution 295 may be administered into the brain using the brain drainage system 210. The concentration of trehalose administered may be approximately a 5 wt % to 40 wt % trehalose solution. The rate of administration of the trehalose solution 295 may be dependent on the metabolism of the individual subject to metabolize the trehalose. In some embodiments, the CSF may be drained simultaneously as the trehalose solution 295 is administered. In some embodiments the administration of the trehalose solution 295 and the drainage of the CSF may be performed in an alternating fashion.

After administration of the trehalose solution 295, the CSF may be sampled periodically or continuously to determine whether a predetermined concentration of trehalose is achieved and/or maintained. The predetermined concentration of trehalose may be associated with a therapeutic concentration range of trehalose in the CSF. For example, the therapeutic concentration range of trehalose in the CSF may be from about 7 wt % to about 10 wt %. If the predetermined concentration of trehalose is not reached, the method 200 includes administering trehalose solution 295 into the brain.

If the predetermined concentration of trehalose is reached, the method 200 includes measuring the CSF for biomarkers. The method 200 includes determining whether a predetermined biomarker concentration in the CSF is reached. The predetermined biomarker concentration may indicate that, in the CSF, there is either an acceptable amount or no amount of inflammation, infection, blood metabolites, or any combination thereof. If the predetermined biomarker concentration is not reached, the method 200 continues to maintain the predetermined trehalose concentration in the CSF and measuring the CSF for biomarkers. The CSF may be periodically or continuously monitored for biomarkers during treatment. If the predetermined biomarker concentration is reached, the trehalose treatment may end. The administration of the trehalose solution may be performed periodically. For example, the trehalose solution may be administered every 30 minutes for the first 48 hours and then adjusted based on the monitoring of the trehalose concentration in the CSF. The duration of trehalose treatment may range from several days to two weeks or more.

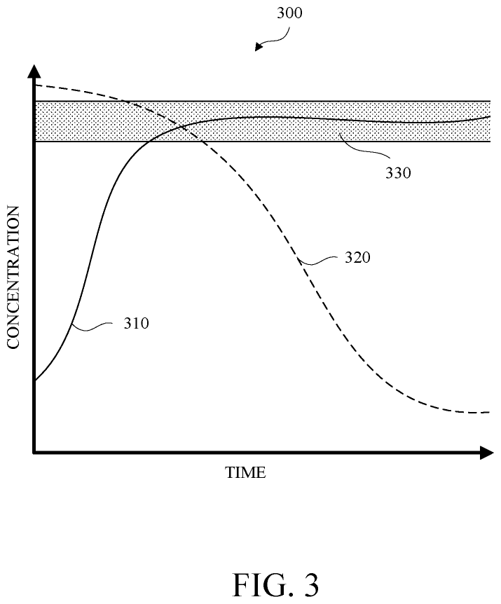

FIG. 3 is a graph 300 showing the concentrations of trehalose 310 and a biomarker 320 during the treatment of vasospasm in accordance with embodiments of this disclosure, for example method 100 of FIG. 1 and method 200 of FIG. 2. As shown in FIG. 3, the concentration of trehalose 310 is low at the beginning of treatment and the concentration of the biomarker 320 is high. As treatment continues, the concentration of trehalose 310 increases until it reaches a therapeutic range 330. The concentration of trehalose 310 is maintained in the therapeutic range 330 until the end of treatment. During the treatment, the concentration of the biomarker 320 reduces until it is undetectable or within an acceptable range. The treatment is ended when the concentration of the biomarker 320 is undetectable or within an acceptable range.

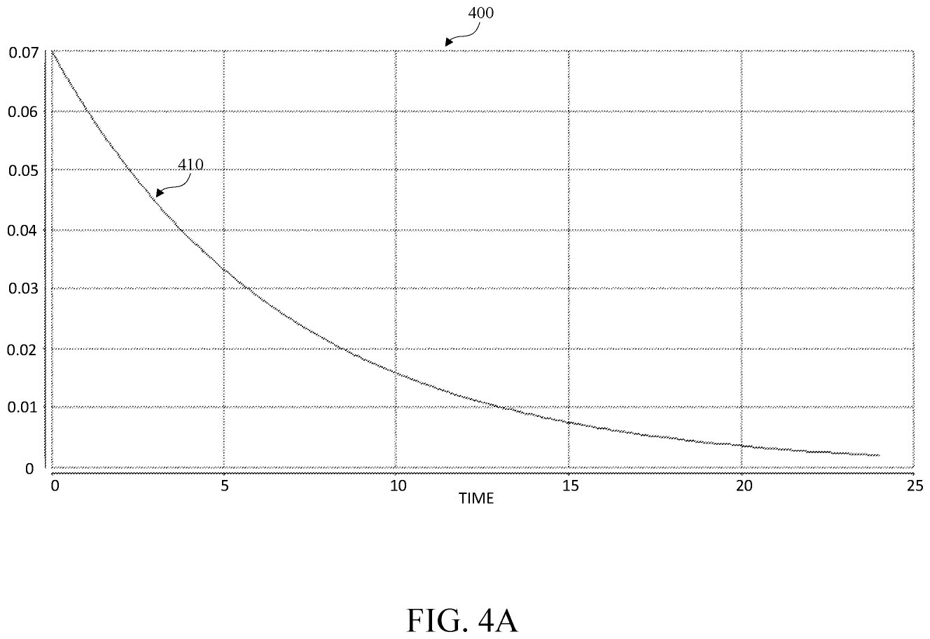

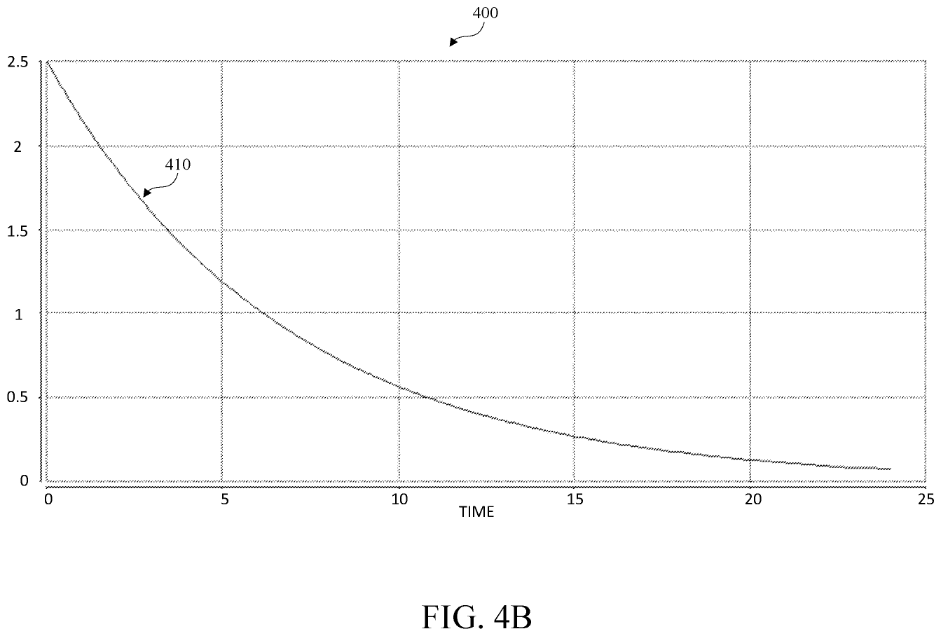

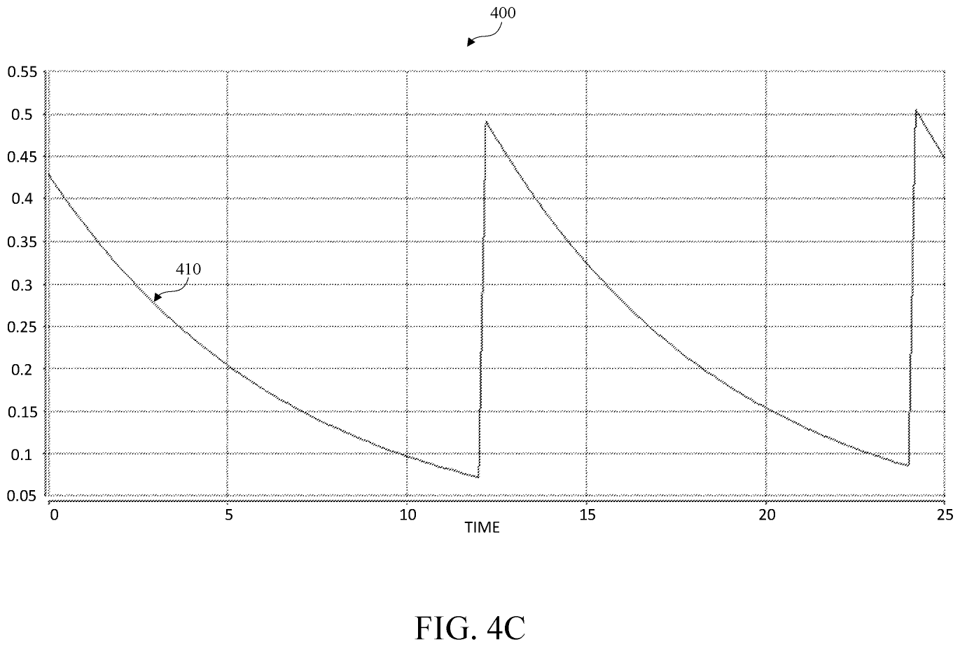

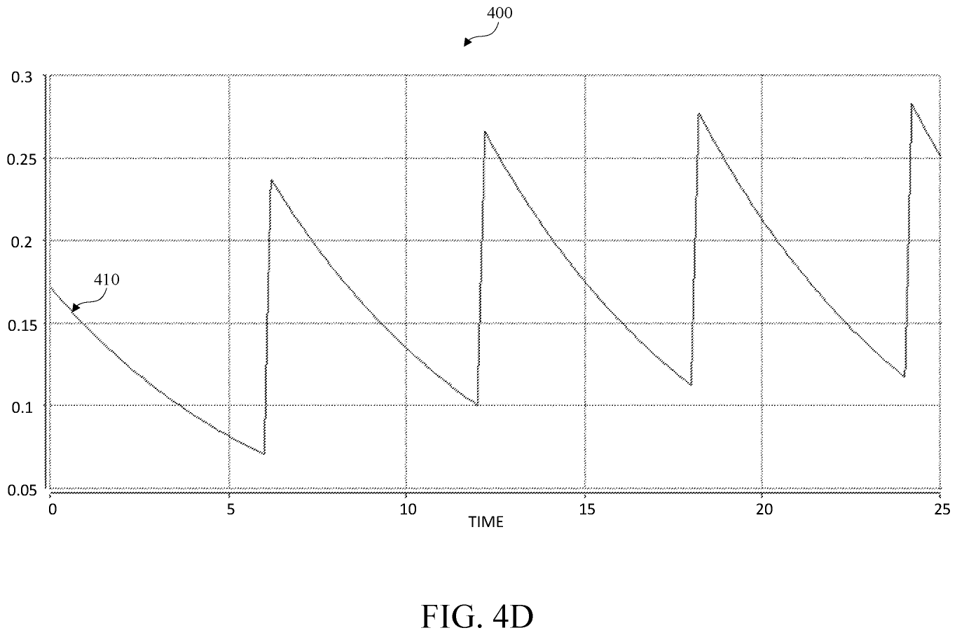

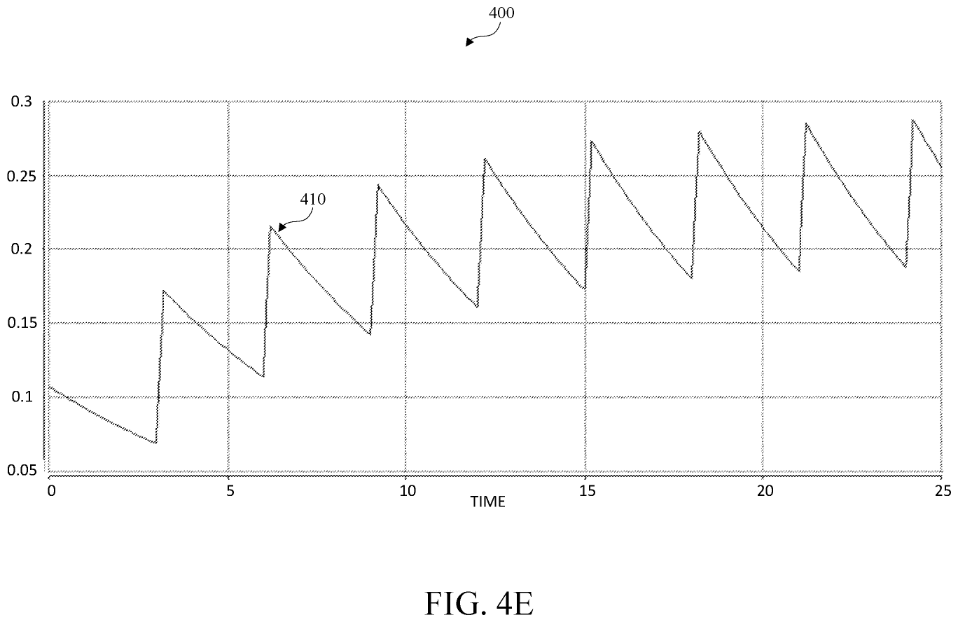

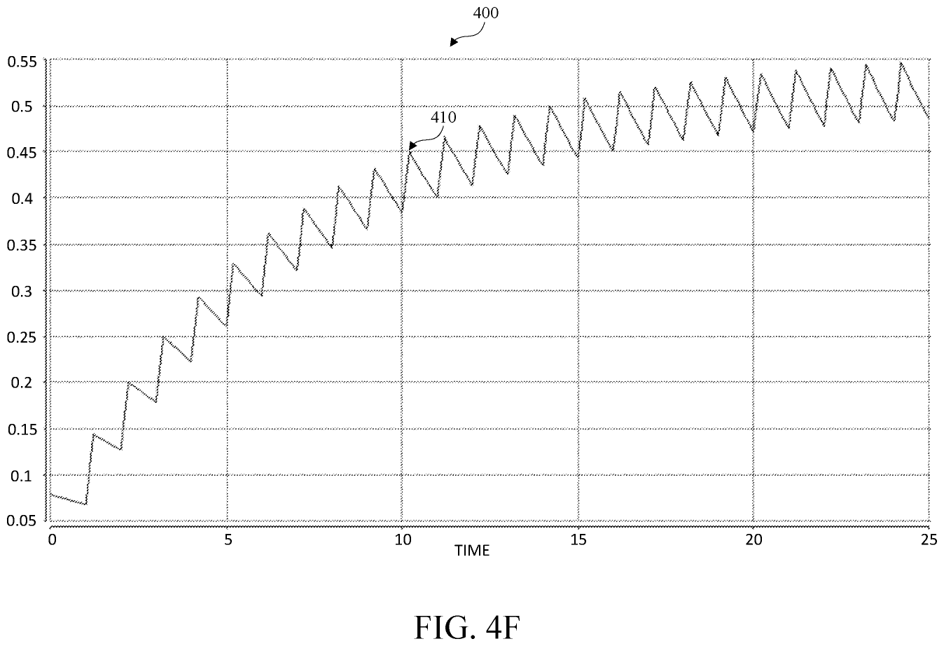

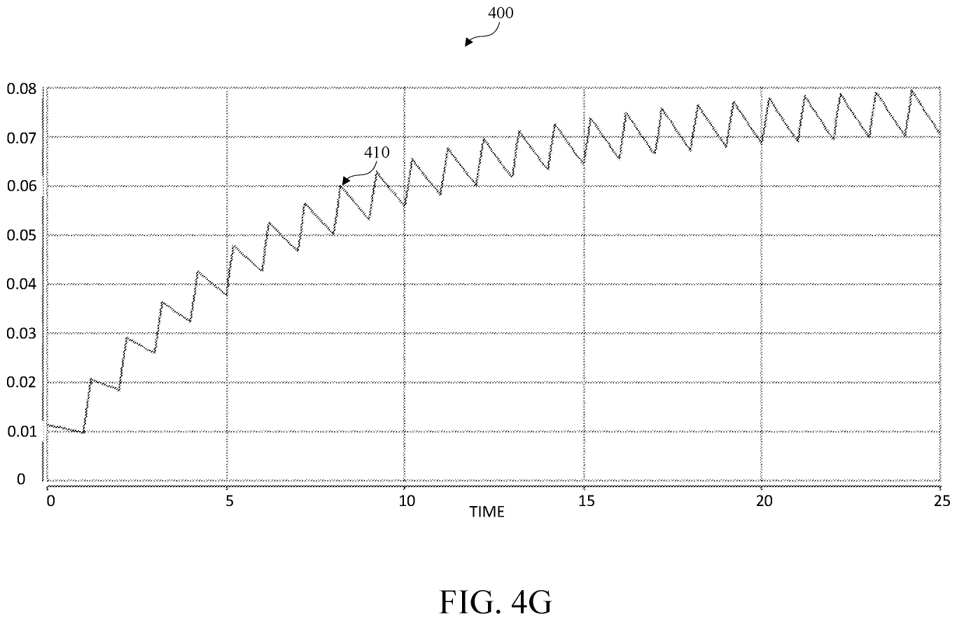

FIGS. 4A-4G are graphs showing simulations of trehalose concentrations in human CSF to determine clinical dose regimens. The simulations assume that a CSF volume of a human is approximately 140 mL and that the CSF exchange rate is approximately 500 mL/hr. Since trehalose does not decompose in the CSF at significant rates, the disappearance of trehalose from the CSF is based on the rate of CSF exchange. In the simulations, the trehalose is administered by a bolus injection, and is equally distributed in the CSF immediately after the injection. For the simulations below, a saturated trehalose solution was prepared by dissolving 68.9 g of trehalose per 100 g of water at 20° C.

FIG. 4A is a graph 400 showing a simulation of trehalose concentration 410 in human CSF to determine a clinical dose regimen. In this example simulation, 9.8 g of trehalose was estimated to be required to achieve a 7 wt % trehalose concentration in the CSF immediately after injection. Since the solubility of trehalose is 0.689 g/mL, approximately 14 mL of saturated trehalose solution was injected. As shown in FIG. 4A, the trehalose concentration 410 rapidly decreases over the course of 24 hours.

FIG. 4B is a graph 400 showing a simulation of trehalose concentration 410 in human CSF to determine a clinical dose regimen. In this example simulation, 350 g of trehalose was estimated to be required to achieve a 7 wt % trehalose concentration in the CSF 24 hours after a single injection. Since the solubility of trehalose is 0.689 g/mL, approximately 500 mL of saturated trehalose solution was injected. As shown in the graph 400, the trehalose concentration 410 dissipates rapidly over the course of 24 hours. Accordingly, this dosing regimen does not have therapeutic value.

FIG. 4C is a graph 400 showing a simulation of trehalose concentration 410 in human CSF to determine a clinical dose regimen. In this example simulation, two injections of 60 g of trehalose was estimated to be required to achieve a 7 wt % or higher trehalose concentration in the CSF using twice daily injections at 12-hour intervals. Since the solubility of trehalose is 0.689 g/mL, approximately 90 mL of saturated trehalose solution was injected for each injection. As shown in FIG. 4C, the trehalose concentration 410 rapidly decreases over the first 12 hours until the second injection. After the second injection, the trehalose concentration 410 spikes and then rapidly decreases again.

FIG. 4D is a graph 400 showing a simulation of trehalose concentration 410 in human CSF to determine a clinical dose regimen. In this example simulation, 24 g of trehalose was estimated to be required per dose. Since the solubility of trehalose is 0.689 g/mL, approximately 35 mL of saturated trehalose solution is estimated for each dose. For the simulation, 35 mL of the concentrated trehalose was administered four times daily at 6-hour intervals to achieve a 7 wt % or higher trehalose concentration in the CSF. As shown in FIG. 4D, the trehalose concentration 410 rapidly decreases over the first 6 hours until the second injection. After the second and subsequent injections, the trehalose concentration 410 spikes and then rapidly decreases again. However, after each injection, the floor of the trehalose concentration 410 slightly increases after each subsequent injection.

FIG. 4E is a graph 400 showing a simulation of trehalose concentration 410 in human CSF to determine a clinical dose regimen. In this example simulation, 15 g of trehalose was estimated to be required per dose. Since the solubility of trehalose is 0.689 g/mL, approximately 22 mL of saturated trehalose solution is estimated for each dose. For the simulation, 22 mL of the concentrated trehalose was administered eight times daily at 3-hour intervals to achieve a 7 wt % or higher trehalose concentration in the CSF. As shown in FIG. 4E, the trehalose concentration 410 rapidly decreases over the first 3 hours until the second injection. After the second and subsequent injections, the trehalose concentration 410 spikes and then rapidly decreases again. However, after each injection, the floor of the trehalose concentration 410 slightly increases after each subsequent injection.

FIG. 4F is a graph 400 showing a simulation of trehalose concentration 410 in human CSF to determine a clinical dose regimen. In this example simulation, 11 g of trehalose was estimated to be required per dose. Since the solubility of trehalose is 0.689 g/mL, approximately 15 mL of saturated trehalose solution is estimated for each dose. For the simulation, 15 mL of the concentrated trehalose was administered twenty-four times daily at 1-hour intervals to achieve a 7 wt % or higher trehalose concentration in the CSF. As shown in FIG. 4F, the trehalose concentration 410 slightly decreases over the first hour until the second injection. After the second and subsequent injections, the trehalose concentration 410 spikes and then slightly decreases again. However, after each injection, the floor of the trehalose concentration 410 slightly increases after each subsequent injection until an approximate steady state is observed. Since the accumulation of trehalose in the CSF is estimated to be observed, the dosage at which the trough concentration of approximately 7 wt % is estimated.

FIG. 4G is a graph 400 showing a simulation of trehalose concentration 410 in human CSF to determine a clinical dose regimen. In this example simulation, 1.6 g of trehalose was estimated to be required per dose. Since the solubility of trehalose is 0.689 g/mL, approximately 2.3 mL of saturated trehalose solution is estimated for each dose. For the simulation, 2.3 mL of the concentrated trehalose was administered twenty-four times daily at 1-hour intervals to achieve a 7 wt % or higher trehalose concentration in the CSF at a steady state. As shown in FIG. 4F, the trehalose concentration 410 slightly decreases over the first hour until the second injection. After the second and subsequent injections, the trehalose concentration 410 spikes and then slightly decreases again. However, after each injection, the floor of the trehalose concentration 410 slightly increases after each subsequent injection until an approximate steady state is observed.

Experimental Results

A first experiment was conducted to establish a procedure of intrathecal (IT) administration to rats to determine the maximum dose volume by IT administration of saline for 30 minutes. An approximately 30 μL of 1.0% Evans blue solution was administered to 6 anesthetized rats via a catheter placed in the subarachnoid space in the cisternal magna. After administration, the animals were necropsied, and the central nervous system (CNS) including the brain and cervical spinal cord were macroscopically observed to verify the distribution of Evans blue solution. In addition, 0.125, 0.25, 0.5, and 1.0 mL of saline, which is considered to be respectively approximately ½, equal, 2-fold, and 4-fold of a total volume of CSF in rats, was administered to 1 or 3 unanesthetized rats per dose volume. Saline was administered for 30 minutes using an infusion pump. Clinical signs were observed during the dosing and until 1 hour after dosing. The animals were necropsied on the next day of dosing and the CNS were macroscopically observed. Macroscopic observation revealed that the Evans blue solution was distributed to the subarachnoid space of the cisterna magna, the bottom of the brain, and the cervical spinal cord in all animals.

Evans blue solution was distributed to the subarachnoid space of the cisterna magna by IT administration, and the bottom of the brain and cervical spinal cord in all the animals was examined Each animal at the dose volumes of 0.5 and 1.0 mL/rat/30 minutes showed tonic convulsions at approximately 12 or 27 minutes after the start of the infusion of saline. In addition, vocalization and abnormal respiratory sounds were observed during an induced convulsion at the dose volume of 1.0 mL/rat/30 minutes. The induced convulsions at the dose volume of 0.5 mL/rat/30 minutes showed an increase in locomotor activity, rolling, tachypnea, escape behavior, and nystagmus in the left eye. An increase in locomotor activity, rolling, and a decrease in locomotor activity were observed in the animals during the infusion of saline at the dose volume of 0.25 and 0.5 mL/rat/30 minutes. By way of contrast, a decrease in locomotor activity was observed transiently during the infusion of saline at the dose volume of 0.125 mL/rat/30 minutes. There were no abnormalities in the brain or cervical spinal cord in any animal at any dose. Based on the above, the maximum feasible dose volume of saline for conscious rats under a restrained condition is approximately 0.125 mL/rat/30 minutes.

A second experiment was conducted to establish a procedure of IT administration to dogs to determine the maximum dose volume by IT administration of saline for 30 minutes. An approximately 1 mL of 0.5% Evans blue solution was administered to 4 anesthetized dogs via a catheter placed in the subarachnoid space near the bregma. After the administration, the animals were necropsied, and the CNS was macroscopically observed to verify the distribution of the Evans blue solution. In addition, 8 or 4 mL of saline, which is considered to be respectively approximately ½ or ¼ of a total volume of CSF, was administered to 6 unanesthetized dogs under a restrained condition. Saline was administered during the dosing and until 1 hour after dosing. The animals were necropsied on the next day of dosing and the CNS were macroscopically observed.

Evans blue solution was distributed to the subarachnoid space of the whole cerebral cortex by IT administration in all 6 animals examined One animal showed tonic convulsions and salivation at approximately 26 minutes after the start of infusion of saline at a dose volume of 8 mL/dog/30 minutes. In the other 5 animals, at the dose volume of 4 mL/dog/30 minutes, there were no clinical signs throughout the 30 minutes of saline infusion. There were no abnormalities in the brain or cervical spinal cord in any animal Based on the above, the maximum feasible dose volume of saline for conscious dogs under a restrained condition is approximately 4 mL/dog/30 minutes.

In all ranges in this disclosure, the endpoints of the ranges are included in the range. While the disclosure has been described in connection with certain embodiments, it is to be understood that the disclosure is not to be limited to the disclosed embodiments but, on the contrary, is intended to cover various combinations, modifications and equivalent arrangements included within the scope of the appended claims, which scope is to be accorded the broadest interpretation so as to encompass all such modifications and equivalent structures as is permitted under the law.

What is claimed is:

1. A method for treating vasospasm, the method comprising:
measuring cerebrospinal fluid (CSF) to obtain a baseline biomarker value;
administering a first dose of a trehalose solution;
draining the CSF to maintain a current intracranial pressure (ICP);
measuring a trehalose concentration in the CSF;
measuring a biomarker value in the CSF; and
on a condition that the measured biomarker value indicates a predetermined biomarker concentration, ending the method for treating vasospasm.

2. The method of claim 1 further comprising:
determining whether a predetermined trehalose concentration is reached; and
on a condition that the predetermined trehalose concentration is not reached, administering a second dose or subsequent of the trehalose solution.

3. The method of claim 1, wherein the administering and the draining are performed simultaneously.

4. The method of claim 1, wherein the administering and the draining are alternately performed.

5. The method of claim 1, wherein the method is performed using a brain drainage system.

6. The method of claim 5, wherein the brain drainage system is a single lumen catheter.

7. The method of claim 5, wherein the brain drainage system is a dual lumen catheter.

8. The method of claim 1, wherein the trehalose solution is approximately a 5 wt % to 40 wt % trehalose solution.

9. The method of claim 1, wherein the measured trehalose concentration in the CSF is within a therapeutic range.

10. The method of claim 9, wherein the therapeutic range is about 7 wt % to about 10 wt %.

11. The method of claim 1, wherein the trehalose solution is administered at a rate based on a metabolism rate of a subject.

12. The method of claim 1, wherein the measured biomarker value is an inflammatory marker value or a blood metabolite value.

13. The method of claim 12, wherein the inflammatory marker value includes a concentration of eukaryotic translation initiation factor 4E (4EBP1), adenosine deaminase (ADA), artemin (ARTN), AXIN1, brain-derived neurotrophic factor (BDNF), beta nerve growth factor (BetaNGF), CASP8, C-C motif chemokine ligand (CCL)11, CCL13/monocyte chemoattractant protein (MCP)4, CCL19, CCL2/MCP1, CCL20, CCL23, CCL25, CCL28, CCL3/macrophage inflammatory protein (MIP)1alpha, CCL4, CCL7/MCP3, CCL8/MCP2, cluster of differentiation (CD)244, CD40, CD5, CD6, CDCP1, colony stimulating factor (CSF)1, CST5, CX3CL1, CXCL1, CXCL10, CXCL11, CXCL5, CXCL6, CXCL9, DNER, EN-RAGE, fibroblast growth factor (FGF)19, FGF21, FGF23, FGF5, FMS-like tyrosine kinase 3 ligand (FLT3L), glial cell line-derived neurotrophic factor (GDNF), hepatocyte growth factor (HGF), interferon (IFN) gamma, interleukin (IL)10, IL10RA, IL10RB, IL12B, IL13, IL15RA, IL17A, IL17C, IL18, IL18R1, IL1alpha, IL2, IL20, IL20RA, IL22RA1, IL24, IL2RB, IL33, IL4, IL5, IL6, IL7, IL8/CXCL8, KITLG/SCF, leukemia inhibitory factor (LIF), LIF receptor (LIFR), lymphotoxin alpha (LTA)/tumor necrosis factor (TNF)B, matrix metallopeptidase (MMP)1, MMP10, neurturin (NRTN), neurotrophin (NTF)3/NT3, oncostatin M (OSM), programmed death-ligand (PDL)1, plasminogen activator urokinase (PLAU)/uPA, SIRT2, signaling lymphocytic activation molecule family member (SLAMF)1, STAMBP, SULT1A1/ST1A1, TGFalpha, TGFB1/, TNF, TNFRSF11B/OPG, TNFRSF9, TNFSF10/TRAIL, TNFSF11/TRANCE, TNFSF12/TWEAK, TNFSF14, thymic stromal lymphopoietin (TSLP), or vascular endothelial growth factor (VEGF)A.

14. The method of claim 12, wherein the blood metabolite value includes a concentration of hemoglobin, biliverdin, carbon monoxide (CO), free ferrous iron (FeII), NF-kB, endothelial cell adhesion molecule (ECAM), vascular cell adhesion molecule-1 (VCAM-1), intracellular cell adhesion molecule-1 (ICAM-1), P-selectin, haptoglobin, hemopexin, or phycocyanobilin related molecules.
